(19) United States
(12) Patent Application Publication
(10) Pub. No.: US 2025/0241257 A1
(43) Pub. Date: Jul. 31, 2025

(54) PHILODENDRON PLANT NAMED ‘DURADO’

(71) Applicant: Robert Roe, Umatilla, FL (US)

(72) Inventor: Robert Roe, Umatilla, FL (US)

(21) Appl. No.: 18/427,498

(22) Filed: Jan. 30, 2024

(51) Int. Cl. A01H 6/10

(52) U.S. Cl. CPC A01H 6/10

(57) ABSTRACT

A new and distinct cultivar of Philodendron Durado named ‘Durado’ particularly characterized by its distinctive leaf appearance; large plant size; mostly open and outwardly spreading fuller plant habit; vigor and slow growth rate; less-freely branching; flat leaves; and long and wide petioles with long petiole sheaths.

### CROSS-REFERENCE TO RELATED APPLICATION

The present application claims priority to U.S. Provisional Patent Application No. 63/441,955 entitled “PHILODENDRON PLANT NAMED ‘Durado’, filed Jan. 30, 2023. The disclosure of which is incorporated in its entirety herein by reference.

### BACKGROUND

The present invention relates to a new and distinct cultivar of Philodendron Durado plant, botanically known as Araceae Philodendron Durado, and hereinafter referred to by the cultivar name “Durado.”

The new Philodendron Durado is a product of an accidental breeding by the Inventor in Umatilla, Florida.

The new Philodendron Durado originated from an accidental breeding made by the Inventor, of an unidentified selection of the Araceae Philodendron Durado. The cultivar was discovered and selected by the Inventor, as a fully grown plant in a 8″ Growers Pot container within the progeny of the self-pollination in a controlled environment in Florida.

Compared to plants of the parent selection, plants of the new Philodendron Durado are larger, branch earlier, are less freely branching, and have a fuller plant habit and longer petioles.

Asexual propagation of the new Philodendron Durado by cuttings has shown that the unique features of this new Philodendron Durado plant are stable and reproduced true to type in successive generations.

### SUMMARY OF THE INVENTION

The new Philodendron Durado has not been observed under all possible environmental conditions. The phenotype may vary somewhat with variations in environment such as temperature, light intensity, fertilizer rate, and/or irrigation amount and frequency without any variance in genotype.

The following traits have been repeatedly observed and are determined to be the unique characteristics of ‘Durado.’ These characteristics in combination distinguish ‘Durado’ as a new and distinct cultivar.
1. Plants of the new Philodendron Durado are self-heading, not vining, and are self-supporting.
2. Plants of the new Philodendron Durado are relatively large in size with a mostly upright, somewhat outwardly spreading and open plant habit.
3. Plants of the new Philodendron Durado are vigorous and have a slow growth rate.
4. Plants of the new Philodendron Durado branch slowly and are early branching.
5. Plants of the new Philodendron Durado have large, thick, smooth, and flat leaves with light green and dark green coloring. The leaves have sporadic green and dark green spots.
6. Plants of the new Philodendron Durado have relatively long petioles and long, green petiole sheaths.

Plants of the new Philodendron Durado can be compared to plants of the commercial Philodendron Durado cultivar Little Hope and Princes of Orange. However, in side-by-side comparisons conducted in Umatilla, Florida, plants of the new Philodendron Durado differ from plants of the cultivar Little Hope and Princes of Orange in the following characteristics:
1. Plants of the new Philodendron Durado are larger and are more outwardly spreading and open than plants of the cultivar Little Hope and Princes of Orange.
2. Plants of the new Philodendron Durado are less vigorous, form branches earlier and are less freely branching than plants of the cultivar Little Hope and Princes of Orange.
3. Leaves of plants of the new Philodendron Durado are thicker, more ovate in shape with truncate bases, and have more numerous primary veins than leaves of plants of the cultivar Little Hope and Princes of Orange.
4. Leaf color of plants of the new Philodendron Durado is darker green and there is a more striking contrast between the mid rib and the surrounding leaf tissue compared to leaves of plants of the cultivar Little Hope and Princes of Orange.
5. Plants of the new Philodendron Durado have longer and wider petioles than plants of the cultivar Little Hope and Princes of Orange. In addition, the shape of the petiole also differs longer, wider, and with green, red, and pink markings.
6. Leaf petiole sheaths of plants of the new Philodendron Durado are greener and darker in color than leaf petiole sheaths of plants of the cultivar Little Hope and Princes of Orange. Plants of the new Philodendron Durado can also be compared to plants of the commercial Philodendron Durado cultivar Little Hope and Princes of Orange. However, in side-by-side comparisons conducted in Umatilla, Florida, plants of the new Philodendron Durado differ from plants of the cultivar Little Hope and Princes of Orange in the following characteristics:
1. Plants of the new Philodendron Durado are larger and are more outwardly spreading and open than plants of the cultivar Little Hope and Princes of Orange.
2. Plants of the new Philodendron Durado are less vigorous, form branches earlier and are less freely branching than plants of the cultivar Little Hope and Princes of Orange.
3. Leaves of plants of the new Philodendron Durado are thicker, more ovate in shape with truncate bases and have more numerous primary veins than leaves of plants of the cultivar Little Hope and Princes of Orange.
4. Leaf color of plants of the new Philodendron Durado is is half yellow-green, half red whereas leaf color of plants of the cultivar Little Hope and Princes of Orange is green. Additionally, there is a more striking contrast between the midrib and the surrounding leaf tissue on plants of the new Philodendron Durado compared to leaves of plants of the cultivar Little Hope and Princes of Orange.
5. Plants of the new Philodendron Durado have longer and wider petioles than plants of the cultivar Little Hope and Princes of Orange. In addition the shape of the petiole also differs.
6. Leaf petiole sheaths of plants of the new Philodendron Durado are greener and lighter in color than leaf petiole sheaths of plants of the cultivar Little Hope and Princes of Orange.
7. Plants of the new Philodendron Durado flower later than plants of the cultivar Little Hope and Princes of Orange.

### BRIEF DESCRIPTION OF THE PHOTOGRAPHS

The accompanying color photographs illustrate the overall appearance of the new Philodendron Durado, “Durado”, showing the colors as true as it is reasonably possible to obtain in color reproductions of this type.

The photographs on sheets one through four comprise top perspective views of a typical plant of “Durado Philodendron” including immature leaves in a (size, e.g. 15 cm) container.

### DETAILED BOTANICAL DESCRIPTION

The following observations, measurements, and comparisons describe plants grown in Umatilla, Florida, under a polycarbonate-covered greenhouse and conditions which closely approximate those used in general, standard commercial horticultural practice. Plants were grown in fertilized soil under day temperatures ranging from 25 to 32 C and night temperatures ranging from 22 to 27 C. Light level ranged from 2000 to 2,2000 footcandles.

Plants used for this description were asexually reproduced by taking cuttings (twigs), rooted and planted into fertilized soil in 15-cm containers. Information was collected for this description and the cultivar comparisons in May 2022, when the plants were about 2 months old from taking cuttings (twigs). Numerical measurements represent averages from typical plants. Environmental conditions were tropical, humid, and required less water than typical Philodendron Durado.

In the following description, color references are made to The Royal Horticultural Society Colour Chart except where general terms of ordinary dictionary significance are used.

Botanical classification: Araceae Philodendron Durado cultivar “Durado Philodendron”.

Parentage: Self-pollination of an unidentified selection of Araceae Philodendron Durado.

Propagation: Type.—By taking cuttings (twigs).

Time to initiate roots.—Summer: About 21 days at about 25 to 30° C. Winter: About 28 days at about 22 to 27° C.

Time to develop (Time to produce a rooted liner).—Summer: About 10 to 12 weeks at about 25 to 30° C. Winter: About 13 to 14 weeks at about 22 to 27 C.

Rooting habit.—Strong root system. Main roots thick; abundant lateral roots. Thick short aerial roots are occasionally observed.

Plant description:

Growth habit.—Self-heading, not vining, and self-supporting. Mostly upright, but outwardly spreading, open, freely branching, Somewhat asymmetrical; mature plants, more rounded in overall shape. Appropriate for 15 to 25-cm containers.

Plant Size.—Height, Soil Surface to top of leaf canopy: About 50.7 cm. Width: About 99.2 cm.

Plant vigor.—Vigorous, slow growth rate.

Crop time.—About 48 to 52 weeks are required to produce a 40 to 50-cm tall plant in a 15-cm container from a 1 to 2-cm taken cutting (twigs).

Foliage description.—Shape of leaves on young plants: Between elliptic and ovate; apex, acuminate with some acute tendencies, base, obtuse; margin, entire. Shape of leaves on older plants: Ovate, apex, acuminate with Some acute tendencies, base, truncate; margin, entire. Length, mature leaves: About 36.7 cm. Width, mature leaves: About 18.8 cm. Length to width ratio, mature leaves: About 1.95 to 1. Aspect: Almost flat; Slightly concave at the petiole attachment. Texture: Thick, leathery, smooth. Surface: Glossy; young leaves very glossy. Color: Young, upper surface: At unrolling: Slightly darker. Once unrolled: Much darker. Young, lower Surface. Mature, upper surface: Darker. About 1 mm wide band along margin, lighter green. Mature, lower surface: Darker and Slightly greener with subsequent development, leaf becomes slightly darker and greener. Venation, upper Surface of mature leaves: Midrib adjacent to petiole with short darker green striae; distal, darker green. Primary veins are slightly lighter green than surrounding leaf tissue. Venation, lower surface of mature leaves: midrib and primary veins, mostly. Petiole aspect: Erect to semi erect. Petiole shape: Rounded lower Surface; upper Surface Slightly concave. Petiole length, primary shot: About 43.4 cm. Petiole diameter, just below leaf blade: About 10.3 mm. Petiole sheath length: About 21.2 cm. Petiole color, young leaves: Short darker green striae. Petiole color, upper Surface of mature leaves: Adjacent to leaf blade. Some proximally darker; slightly lighter near base. Short darker green Striae. Short darker green striae. Petiole sheath: and with short darker green striae, Some light green, present at proximal end near edges, more noticeable at distal end.

Inflorescence.—Typical of Philodendron Durado no commercial significance. Plants typically form inflorescences about 18 to 23 months after planting of a twig taken from cutting. Description of inflorescences taken from plants grown for about 2.5 years after planting a twig taken from cutting.

Arrangement.—Spathes with spadices with one to three inflorescences in each floral sympodium. Each developing inflorescence enclosed by two sheaths, which deteriorate before spathe opens. Spathes erect, situated near shoot base on short, thick, usually bent peduncles. Spathes typically tightly tolled around spadices, Spathes typically open for about one day. When spathes are open, the spadix is fully exposed, leaning forward beyond spathe margins, at about 50 to penduncle axis.

Fragrance.—weak, Similar to other Philodendrons, but weaker.

Peduncle.—Length: About 7.3 cm. Diameter: About 1.5 cm.

Spathe.—Shape: Boat-shaped; longitudinal section elliptic; apex between acuminate and cuspidate; base, obtuse; margin, entire and usually rolled back especially distally. Length: About 18.3 cm. Width: About 7.6 cm. Length to width ratio: About 2.4 to 1. Depth: About 4.8 cm. Texture: Very thick, fleshy, not glossy. Color: Open, front surface: Around spadix base, distally and toward margin, much lighter, near apex with some scattered light green and dark green. Some light green on midrib and along margins near apex. Open, rear surface: Most of the central Zone of proximal half is an uneven color. Color becomes lighter towards margins, near margins, and translucent. In the distal half, there is a band along midrib. Spathe color on either side of this band is variable, but much lighter green Often there is a wide, translucent band on one side near the margin.

Spadix.—Shape: Cylindrical, Strongly tapering towards acute apex. Female Zone, Situated at the base, shorter than the male Zone and Separated by an intermediate Sterile Zone. Sterile and male Zones not clearly distinguished when Spathe is open. Spadices typically sessile. Length: About 17.1 cm; female Zone, about 4.8 cm; Sterile and male Zones combined, about 12.6 cm.

Diameter: Female Zone, about 1.9 cm; sterile and male zones, about 1.8 cm proximally. Color: Female Zone, sterile and male zones, slightly darker. Pollen: Cream in color. Dehiscence about one to three days after spathe closes.

Seed production.—Seed production has not been observed. Typically spathes and spadices gradually deteriorate after spathe closure.

Disease tolerance: Resistance to pathogens common to Philodendron Durado has not been observed.

What is claimed is:

1. A new and distinct cultivar of Philodendron Durado plant named ‘Durado’, as illustrated and described.
